(19) United States
(12) Patent Application Publication
(10) Pub. No.: US 2026/0285690 A1
(43) Pub. Date: Sep. 24, 2026

(54) CLEANING OF CO2 CONTAINING FEED GASES

(71) Applicant: TOPSOE A/S, Kgs. Lyngby (DK)

(72) Inventors: Martin ØSTBERG, Kgs. Lyngby (DK); Birgitte Staal HAMMERSHØI, Kgs. Lyngby (DK); Mads Kristian KAARSHOLM, Kgs. Lyngby (DK); Morten THELLEFSEN, Kgs. Lyngby (DK)

(73) Assignee: TOPSOE A/S, Kgs. Lyngby (DK)

(21) Appl. No.: 19/476,029

(22) PCT Filed: Apr. 19, 2024
(86) PCT No.: PCT/EP2024/060763

§ 371 Date: Oct. 16, 2025

(30) Foreign Application Priority Data
Apr. 21, 2023 (DK) 202300340

(51) Int. Cl. C01B 32/50; B01D 53/04; B01D 53/26; C07C 41/01; C10G 2/00

(52) U.S. Cl. CPC C01B 32/50; B01D 53/04; B01D 53/265; C07C 41/01; C10G 2/30; B01D 2256/22; B01D 2257/104; B01D 2257/302; B01D 2257/304; B01D 2257/306; B01D 2257/80; C01P 2006/80

(57) ABSTRACT

The present invention relates to a process for cleaning a CO₂-rich gas feed, in the presence of a hydrogen-rich feed, in particular for removing sulfur-containing impurities, and optionally oxygen.

### TECHNICAL FIELD

The present invention relates to a process for cleaning a CO₂-rich gas feed, in particular for removing sulfur-containing impurities, and optionally, oxygen (O₂).

### BACKGROUND

Carbon dioxide is commercially available in different grades. Typically, “food grade” or “beverage grade” CO₂ has a purity of 99.9%. However, for processes involving catalytic conversion of CO₂ to other chemical products (e.g. power-to-X), impurities such as sulfur-containing impurities and oxygen in the CO₂ stream may poison the catalyst, even when present at concentrations of 0.0001 vol %.

Despite the high purity of certain CO₂ sources, it has been discovered that further purification is required to avoid catalyst poisoning of downstream synthesis.

Sulfur compounds are well-known as catalyst poisons, which react with the active material on the catalysts and render them catalytically inactive. For some catalysts, oxygen (O2) is also such a poison and e.g. the Cu-based methanol catalyst is prone for degradation by oxygen and therefore any oxygen present in the CO2 (or H2) feed gases to a methanol plant has to kept at low concentration and in some cases it has to be removed from the feed gases.

The catalyst/absorbent systems developed to remove sulfur impurities from CO2 have been found to be sensitive to relatively high oxygen concentrations as well, and a solution has been developed to remove the O2 from the CO2 gas before the CO2 gas enters the sulfur removal systems.

Systems and processes for purification of CO2 streams are known from e.g. EP2457636, CN112999843 and CN112957872.

### SUMMARY

It has been found by the present inventor(s) that cleaning of CO₂ feeds is necessary and can be carried out by adsorption of sulfur containing impurities on a metal-promoted guard material, with conversion of SO₂ or organic-bound sulfur to H₂S prior to adsorption. It has also been discovered that any oxygen in the CO₂ feed can influence the sulfur capacity of a guard material and catalytic activity of a sulfur hydrogenation catalyst.

So, in a first aspect the present invention relates to a process for cleaning a CO₂-rich gas feed, said CO₂-rich gas feed comprising at least 80 wt % CO₂ and one or more sulfur-containing impurities; wherein said process comprises the step of:
passing the CO₂-rich gas feed together with a hydrogen-rich feed over a first catalyst active in hydrogenation, whereby at least a portion of the SO₂ or organic-bound sulfur in the CO₂-rich gas feed is converted to H₂S, to thereby provide a H₂S-containing CO₂ gas stream,
passing the H₂S-containing CO₂ gas stream over one or more guard material(s), and adsorbing H₂S and optionally one or more sulfur-containing compounds on a guard material, to provide a cleaned CO₂-rich gas stream.

A process for production of a syngas stream is also provided, said process comprising the process described above, and further comprising:
providing at least a portion of said cleaned CO₂-rich gas stream from the process described above;
providing a second hydrogen-rich feed, optionally obtained from the process of electrolysis of water in one or more electrolysis unit(s);
reacting at least a portion of said portion of the cleaned CO₂-rich gas stream with the second hydrogen-rich feed, to provide at least one syngas stream.

A process is also provided for production of a synthetic fuel stream, said process comprising the process described above, and further comprising the step of converting said at least one syngas stream to at least one synthetic fuel stream, preferably being a MeOH stream, a DME stream, or a synthetic fuel stream, fuels being aviation fuel, gasoline, diesel or similar.

Additional aspects are presented in the following description text, figures and claims.

### LEGENDS

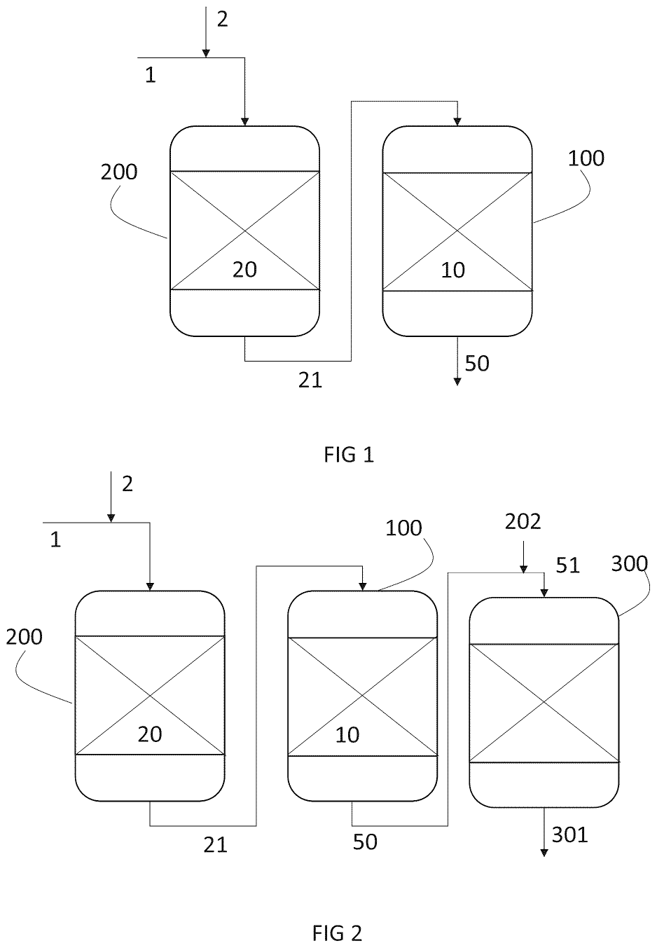

FIG. 1 shows a simple layout of one embodiment of the process of the invention.

FIG. 2 shows a layout for production of a synthetic fuel stream.

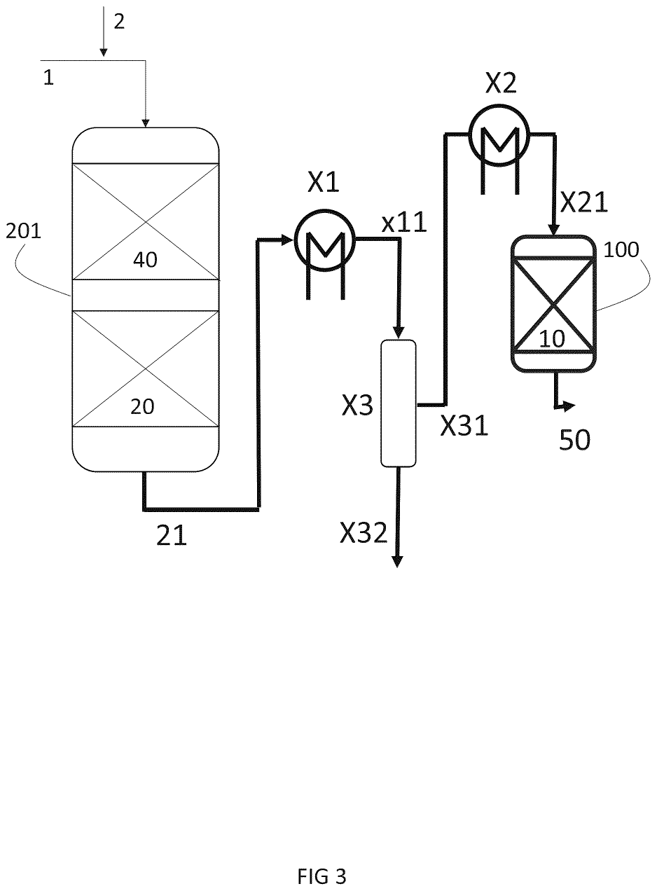

FIG. 3 shows a layout of one embodiment of the process of the invention in which there is an optional water removal step downstream the hydrogenation catalyst(s)

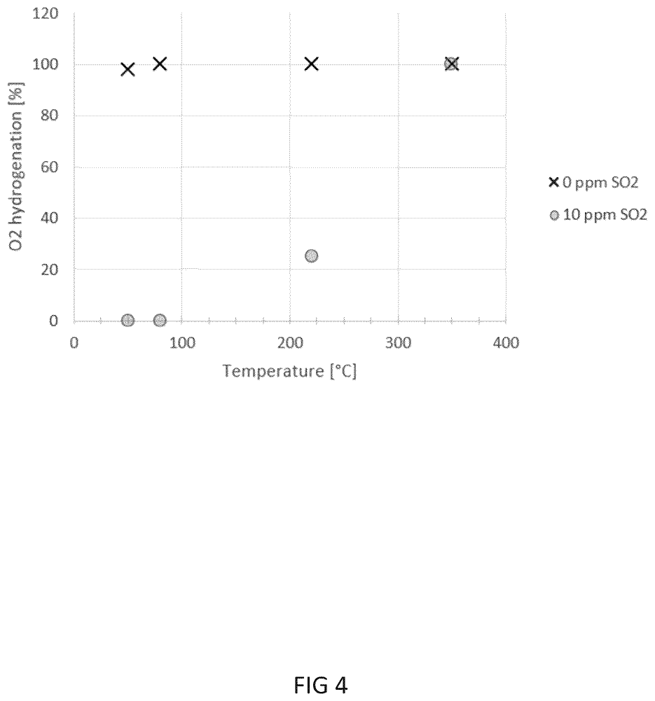

FIG. 4 show the oxygen hydrogenation efficiency as a function of temperature and SO₂ concentration for an oxygen hydrogenation catalyst.

### DETAILED DISCLOSURE

Unless otherwise specified, any given percentages for gas content are % by volume. All feeds are preheated as required.

Syngas is used as reference for a synthesis gas, a gas mixture comprising hydrogen, carbon monoxide, carbon dioxide and typically water as steam and methane. It is referred to as syngas/synthesis gas because it is the feed for a downstream catalytic synthesis leading to the desired product. In some application the feed downstream the referred purification can be mixed with hydrogen and be used as synthesis gas e.g. for methanol synthesis in other applications, the purified gas may after mixing with hydrogen and optionally steam need conversion in a reverse water gas shift reactor (RWGS) or combined RWGS and methanation reactor to form the final synthesis gas for the synthesis of the final product.

A cleaned CO₂ stream is defined as the outlet stream from the CO₂ cleaning process, in which minimum 95% of the combined sulfur containing impurities in the feed is removed or the sum of sulfur containing impurities in the clean CO₂ stream is lower than 500 ppbv (parts per billion by volume), preferably lower than 100 ppbv and most preferably lower than 50 ppbv.

The sum of sulfur containing impurities should be understood as sulfur equivalents, i.e. 10 ppbv SO₂ correspond to 10 ppbv sulfur and 10 ppbv CS₂ correspond to 20 ppbv sulfur

Similarly, cleaned CO₂ stream is defined as the outlet stream from the CO₂ cleaning process, in which minimum 95% of the oxygen in the feed is removed or the O₂ concentration in the clean CO₂ stream is lower than 200 ppmv (parts per million by volume), preferably lower than 100 ppmv and most preferably lower than 50 ppmv.

The proposed CO₂ cleaning solution ensures that the feed gases for any downstream conversion to synthesis gas and synthesis for chemicals like MeOH (methanol), DME (dimethyl ether), FT (Fischer Tropsch), synthetic fuels etc. can be made without poisoning of the downstream synthesis catalyst involved by sulfur. This will ensure that operation can be made over time and allow catalyst lifetime as expected for industrial catalyst.

In a first aspect, therefore, a process for cleaning a CO₂-rich gas feed is provided.

The CO₂-rich gas feed provided to the process comprises at least 90 wt % CO₂, such as at least 95 wt % CO₂, such as at least 99.0 wt % CO₂, preferably at least 99.5 wt % CO₂, more preferably as at least 99.9 wt % CO₂. The CO₂-rich gas feed is thus already of high purity prior to the process of the present invention.

Suitably, the CO₂-rich gas feed is derived from a renewable source, such as:
combustion or gasification of a lignocellulosic biomass such as wood products, algae, grass, forestry waste and/or agricultural residue;
combustion or gasification of municipal waste, in particular the organic portion thereof, where the municipal waste is defined as a feedstock containing materials of items discarded by the public, such as mixed municipal waste given in EU Directive 2018/2001 (RED II), Annex IX, part A;
microbial conversion of nitrogen-rich renewable feedstock such as manure or sewage sludge;
fermentation of hydrocarbon (sugar) rich feed streams such as corn, sugar cane and beets.

The CO₂-rich gas feed can also be obtained from direct air capture processes, metallurgical processes, cement production or fossil fuel combustion.

The CO₂ concentration in some of the above-mentioned gas streams may typically be too low for further chemical processing and a concentration step is required to increase the CO₂ concentration to the desired value as mentioned above.

The CO₂-rich gas feed comprises one or more sulfur-containing impurities. The one or more sulfur-containing impurities within the CO₂-rich gas feed may be selected from organosulfur compounds such as thiols, sulfides, disulfides, sulfones, sulfoxides and thioketones, CS₂, COS, SO₃, SO₂ and H₂S, preferably H₂S and SO₂, most preferably SO₂. The total content of SO₂ in the CO₂-rich gas feed is 0.1-50 ppm V SO₂, such as 0.2-10 ppm SO₂, such as 1-10 ppm V SO₂, such as 0.5-5 ppm SO₂, such as 1-5 ppm V SO₂.

The CO₂-rich gas feed may also comprise water and water may be formed by the first catalyst active in hydrogenation. It is desirable, however, to keep the water content as low as possible as it affects the equilibrium concentration of hydrogen sulfide in the gas phase, also called the H₂S slip. Suitably, therefore, the total content of H₂O in the H₂S-containing CO₂ gas stream is no more than 10 vol %, preferably no more than 5.0 vol %.

Hydrogen sulfide reacts with ZnO contained in the guard forming zinc sulfide and the amount of hydrogen sulfide that can be removed from the gas phase is dependent on the water concentration according to the following chemical equilibrium:

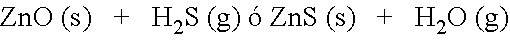

The H₂S slip from the guard material will increase with water concentration and temperature.

The CO₂-rich gas feed may—in certain cases—comprise oxygen (O₂). Oxygen may also contaminate, poison or lead to degradation of downstream catalysts and guard material, so any oxygen in the CO₂-rich gas feed should often be reduced or eliminated. The total content of O₂ in the CO₂-rich gas feed is 50-10,000 ppm O₂, such as 50-5000 ppm O₂, such as 100-1000 ppm O₂.

The CO₂ rich gas feed may comprise other impurities such as nitrogen oxides (NOx) or alkenes or dienes, alcohols, aldehydes etc. These contaminants may also be hydrogenated over the first hydrogenation catalyst.

Generally, the process comprises the step of: passing the CO₂-rich gas feed together with a hydrogen-rich feed over a first catalyst converting the sulfur-containing impurities (particularly SO₂) to H₂S and subsequently adsorbing H₂S and optionally one or more sulfur-containing compounds on one or more guard material(s), to provide a cleaned CO₂-rich gas stream. The guard material may be a ZnO, a Cu-promoted ZnO or a Cu—Zn—Al type. The first catalyst is suitably a CoMo or NiMo type catalyst active in hydrogenation of the feed stream including hydrogenation of SO₂ to H₂S and H₂O and other organic sulfur compounds into H₂S and COS. The active form of the NiMo or CoMo catalyst is in the sulfided state and a presulfidation is necessary to activate the first catalyst for hydrogenation.

The hydrogenation of SO₂ proceeds according to the overall reaction

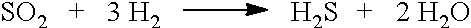

The CO₂-rich gas feed to be purified is first mixed with a hydrogen-rich feed, which acts as a reductant for one or more sulfur-containing impurities in the CO₂-rich gas feed. The hydrogen-rich feed to the process comprises at least 90 wt % hydrogen such as at least 95 wt % hydrogen such as at least 98 wt % hydrogen. Hydrogen is suitably added such that, the total content of H₂ in the combined feed of the CO₂-rich gas feed and the hydrogen-rich feed, after mixing of said feeds, is 0.2-10 vol % H₂ such as 0.5-3 vol % H₂. The addition of H₂ is controlled to limit undesired side reactions, e.g. water and/or methanol formation while providing sufficient reduction potential for the desired conversion of sulfur compounds. The addition of hydrogen should always be sufficient to achieve excess H₂ in the product gas exiting the hydrogenation reactor.

Undesired hydrogenation reactions could be the reverse water gas shift reaction and the methanol formation reaction:

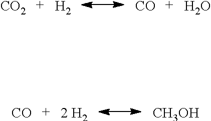

The one or more sulfur-containing compounds adsorbed onto the guard material is typically H₂S.

The operating pressure of the first catalyst and adsorption system will be in the range 1 to 90 barg, depending on the feed pressure of the CO₂ and H₂ feed gases and the pressure of the downstream synthesis plant. Generally, a lower pressure will be more beneficial with regard to minimizing the formation of undesired side products, but it comes with a cost of larger equipment.

The operating temperature of the first hydrogenation catalyst is typically in the 250-450° C. range, which represent a compromise between catalytic activity and the formation of undesired side products.

The operating temperature of the guard materials is in the 80-450° C. range, which represent a compromise between adsorption capacity, adsorption rate and H₂S slip.

The first hydrogenation catalyst material is typically a so-called NiMo or CoMo type, with a chemical composition of 1-5% w/w nickel or cobalt, 5-20% w/w molybdenum and 75-94% w/w alumina.

The sulfur adsorption guards can be e.g., an almost pure ZnO material, a Cu-promoted ZnO or a Cu—Zn—Al type with a composition of 25-60% w/w Cu, 15-30% w/w Zn, 2-10% w/w Al. The guard material may be present as oxides or basic oxides.

The oxygen hydrogenation catalysts, typically comprising an alumina or silica carrier impregnated with small amounts of active (noble) metals, such as Cu, Mn, Pt and/or Pd are very efficient for the O₂+2 H₂->2H₂O reaction and high conversion can be achieved at temperatures as low as 50° C. in gases free of sulfur compounds. However, these catalysts are prone to sulfur poisoning at low temperatures and the practical choice is then to operate the oxygen hydrogenation catalyst at higher temperatures, where the sulfur poisoning does not take place. This can be implemented relatively easy as the sulfur hydrogenation catalyst has an operating temperature range, which overlap with the 250-400° C. temperature range for the oxygen hydrogenation catalyst. As shown in FIG. 1, the CO₂-rich gas feed together with a hydrogen-rich feed is passed over a first catalyst, being active in hydrogenation of SO₂ to H₂S, whereby at least a portion of the SO₂ or organic-bound sulfur in the CO₂-rich gas feed is converted to H₂S to provide a H₂S-containing CO₂ gas stream, followed by the step of passing said H₂S-containing CO₂ gas stream over said one or more guard material(s), where at least a fraction of the H₂S is adsorbed.

In this aspect, therefore, the guard material is active in absorption of H₂S, and is preferably a ZnO or Cu-promoted ZnO guard material. The guard may also be a Cu—Zn—Al type.

According to one aspect, adsorption onto the guard material can be a two-step process. In this manner, a second guard material may be used to remove any unconverted and unadsorbed sulfur-containing impurities. According to this aspect, the process comprises:
Passing the H₂S-containing CO₂ gas stream over a first sulfur guard material, followed by
passing the H₂S-containing CO₂ gas stream over a second sulfur guard material and adsorbing one or more sulfur-containing compounds on said first and said second sulfur guard materials, to provide a cleaned CO₂-rich gas stream.

Preferably, the first sulfur guard material is characterized by being prone to H₂S slip, while the second sulfur guard material, is characterized by not exhibiting H₂S slip. Optionally, water may be removed from the CO₂ gas stream between first and second guard materials.

The first and second sulfur guard materials can have the same composition, wherein the second sulfur guard material is at different process conditions which allow for no H₂S slip operation. That could be accomplished by installing a water removal unit between the first and second sulfur guard material and/or lowering the temperature between the first and second sulfur guard material by installing a heat exchange unit.

The second sulfur guard material can have a chemical composition different from the first sulfur guard material, e.g. a ZnO based first guard material and a Cu—Zn—Al based second sulfur guard material. Even so it may be beneficial to operate the two guard materials at different process conditions as suggested above.

For both a single and multiple guard material layout, it may also be beneficial to change operating conditions between the first hydrogenation catalyst and guard material(s).

As a further option, the H₂S-containing CO₂ gas stream is passed over an optional chlorine guard material active to remove chlorine, prior to being passed over the first guard material. The chlorine guard material may be an alkali metal (preferably K or Na) supported on alumina.

The best layout and operation of the CO₂ cleaning section will depend on the process conditions, the guard adsorption properties, desired CO₂ purity and cost of installing extra vessels and equipment.

In the two-stage process illustrated in FIG. 1, the first step will typically convert any SO₂ present in the feed to H₂S and water. Therefore—in this aspect—the process optionally comprises a step of removing water from the H₂S-containing CO₂ gas stream, prior to passing it over said guard material. Water removal may e.g. take place via cooling and/or compression of the H₂S-containing CO₂ gas stream.

In an embodiment, the CO₂-rich gas feed additionally comprises oxygen (O₂), and wherein process further comprises the step of passing the CO₂-rich gas feed together with a hydrogen-rich feed over a second catalyst active in oxygen hydrogenation, and being positioned upstream the first catalyst, thereby converting at least a portion of the oxygen in the CO₂-rich gas feed to H₂O.

If the concentration of O₂ in the CO₂ rich gas feed is relatively low, the same catalysts and guard materials will thus be able to remove O₂ by hydrogenation, at the same time as removing sulfur-containing impurities. In such situations, the first catalyst and the second catalyst may be the same. However, if the O₂ concentration in the CO₂ rich gas feed is relatively high, it will affect the first NiMo or CoMo based hydrogenation catalyst and the oxygen must be reduced or removed before the sulfur species is hydrogenated. As this leads to an increase of the H₂O concentration, it may be advantageous to remove H₂O from the gas leaving the first hydrogenation catalyst before it contacts the guard material. Therefore, in one embodiment, the process further comprises a step of removing H₂O from the CO₂-rich gas feed, prior to the step of passing the H₂S-containing CO₂ gas stream over one or more guard material(s), and adsorbing H₂S and optionally one or more sulfur-containing compounds on a guard material. The step of removing H₂O suitably comprises cooling the CO₂-rich gas feed, e.g. in a feed effluent heat exchanger. This option is also suitable if the downstream synthesis could occur at a lower temperature e.g. methanol synthesis.

The process provides a cleaned CO₂-rich gas stream. This cleaned CO₂-rich stream typically comprises:
less than 500 ppb, preferably less than 100 ppb and most preferably less than 50 ppb sulfur in all forms
less than 200 ppmv O₂, less than 100 ppmv O₂, less than 50 ppmv O₂, less than 10 ppm O₂, less than 5 ppm O₂, less than 1 ppm O₂

The first hydrogenation catalyst is suitably located within a reactor vessel, said reactor vessel being arranged to receive the CO₂-rich gas feed and the hydrogen-rich feed, optionally in admixture. The guard material used in the process of the invention is suitably also located within a separate reactor vessel although—in certain circumstances—it may be possible or desirable to arrange both the guard material and the first hydrogenation catalyst in the same reactor vessel.

The CO₂ cleaning process is typically operated in the pressure range 1-90 bar, preferably 1-50 bar, depending on the pressure of the CO₂-rich feed stream and the pressure of the downstream conversion process.

As noted above, the cleaned CO₂-rich gas stream is sufficiently pure that catalyst poisoning of downstream processes is significantly reduced. The invention therefore provides a process for production of a syngas stream, said process comprising the process as described above, and further comprising:
providing at least a portion of said cleaned CO₂-rich gas stream from the process described herein;
providing a second hydrogen-rich feed, optionally obtained from the process of electrolysis of water in one or more electrolysis unit(s);
mixing at least a portion of said portion of the cleaned CO₂-rich stream feed (50) with the second hydrogen-rich feed (202), to provide at least one syngas feed stream (51);
passing the syngas feed stream over one or more catalyst(s) active in converting CO₂ and H₂ into a mixture of CO, H₂O, H₂ and CO₂

An integrated process can also take place, in which CO₂ cleaning, syngas production and subsequent downstream syntheses occur. Therefore, a process for production of a synthetic fuel stream is provided, said process comprising providing at least one syngas stream, as described herein, and further comprising the step of converting said at least one syngas stream to at least one synthetic fuel stream, preferably being a MeOH stream, a DME stream, or a synthetic fuel stream.

### Specific Embodiments

FIG. 1 shows an embodiment of the process of the invention. A CO₂-rich gas feed 1 is mixed with a hydrogen-rich feed 2 and passed over a first catalyst 20 active in hydrogenation of SO₂ and organic bound sulfur to H₂S in a first reactor 200. At least a portion of the SO₂ or organic-bound sulfur in the CO₂-rich gas feed 1 is converted to H₂S to provide a H₂S-containing CO₂ gas stream 21. This conversion is followed by passing the H₂S-containing CO₂ gas stream 21 over guard material 10 where at least H₂S is adsorbed in a second reactor (100). A cleaned CO₂-rich gas stream 50 is outputted.

The first reactor (200) and the second reactor (100) may be combined in one reactor with two beds (10) and (20).

FIG. 2 shows a layout for production of a synthetic fuel stream 301, in a synthesis section 300. The reactor vessels 100, 200, CO₂-rich gas feed 1, hydrogen-rich feed 2 and cleaned CO₂-rich gas stream 50 are according to FIG. 1. A second hydrogen-rich feed 202 is mixed with the cleaned CO₂-rich stream feed 50, to provide a syngas stream 51. This syngas stream 51 is converted to at least synthetic fuel stream 301 in a synthesis section 300.

FIG. 3 shows a layout for production of a cleaned CO₂-rich gas (50). A CO₂-rich gas feed 1 is mixed with a hydrogen-rich feed 2 and passed over a second catalyst 40 active in hydrogenation of O₂ and subsequently over a second catalyst active in hydrogenation of SO₂ and organic bound sulfur to H₂S (20) in first reactor 201. At least a portion of the SO₂ or organic-bound sulfur in the CO₂-rich gas feed 1 is converted to H₂S to provide a H₂S-containing CO₂ gas stream 21. The H₂S and H₂O-containing CO₂ gas stream 21 is optionally cooled in a heat exchanger (X1) to condense H₂O separating the stream in a separator to a H₂O depleted H₂S-containing CO₂ gas stream (X31) and a water stream (X32). The H₂O depleted H₂S containing CO₂ gas stream (X31) is heating in a heat exchanger (X2), where part of the required cooling/heat in X1 and X2 may be combined in a feed-effluent heat exchanger. The heated H₂O depleted H₂S-containing CO₂ gas stream (X21) is depleted in sulfur species over guard material 10 where at least H₂S is adsorbed. A cleaned CO₂-rich gas stream 50 is outputted.

The reactor (201) with (40) and (20) may be split in two separate reactors and may be operated at the same temperature or at different temperatures if process conditions will be more favourable. The hydrogenation of O₂ is an exothermal reaction and can increase the temperature of the gas mixture by up to 100-110° C. per vol % of O₂ hydrogenated, such that the adiabatic temperature increase of a CO₂-rich gas with 5,000 ppm O₂ (0.5 vol %) and 4 vol % H₂ will be around 55° C. This temperature increase can be used to preheat the CO₂ gas to the sulfur hydrogenation catalyst, e.g. by operating with O₂ hydrogenation catalyst with a 295° C. inlet temperature to obtain the desired 350° C. inlet temperature to the sulfur hydrogenation catalyst. It may also be necessary to cool the process gas between the two catalysts or even supply more heating between the two catalysts. Heat exchangers, heaters and coolers must then be installed between the catalysts.

### Examples

Experiments have been carried out in a laboratory fixed bed reactor at isothermal condition to test the conversion of SO₂ to H₂S using H₂ as the reductant. The fixed bed reactor is placed in an electrically-heated oven and heated to the desired operating temperature. Two internal thermocouples measure the inlet and exit temperatures in the catalytic bed. The oven is equipped with external thermocouples controlling the temperature zones in the oven. These zones are controlled by internal thermocouple readings to obtain isothermal reaction condition in the fixed bed.

The first hydrogenation catalyst material is placed in a SilcoNert 2000™ coated stainless steel reactor on a grid and the reactor is aligned to be in the center of the electrical oven. Feed gases are mixed from gas cylinders using mass flow controllers controlling feed of the individual gases. These include nitrogen (N₂), hydrogen (H₂), carbon dioxide (CO₂), and 15 ppm sulfur dioxide in methane (SO₂ in CH₄).

The first hydrogenation catalyst material used is NiMo-impregnated alumina 1/10″ Trilobe extrudates. In the individual test, the catalyst was mixed before loading with catalytically inert carborundum 46, mesh 60 to give a loading height of 110 mm. The catalyst dilution was carried out to limit the chemical conversion such that catalytic activity data could be acquired.

The NiMo-impregnated catalyst was activated at 350° C. by flushing with a gas at 30 barg containing 1.1 ppm SO₂, 2.5% H₂, 7.5% CH₄ and 90.0% CO₂. Table 1 lists the different test condition carried out with this gas and the sulfur exit analysis obtained after obtaining stable exit concentrations at each test condition.

TABLE 1
Load of 0.5 g NiMo/alumina extrudates which have been sulfided
for 200 hours with 1 ppm SO₂ prior to the experiments.

| | Conditions | | | Feed composition | | | | Exit composition measured | | | |
|---|---|---|---|---|---|---|---|---|---|---|---|
| | Temp, | P, | Flow, | H₂, | CO₂, | CH₄, | SO₂, | SO₂, | DMS, | H₂S, | COS, |
| Test# | ° C. | barg | Nl/h | % | % | % | ppm | ppm | ppm | ppm | ppm |
| 1a | 350 | 30 | 40 | 2.5 | 90.0 | 7.5 | 1.177 | <0.05 | 0 | 0.742 | 0.127 |
| 1b | 340 | 30 | 40 | 2.5 | 90.0 | 7.5 | 1.170 | <0.05 | 0.040 | 0.743 | 0.119 |
| 1c | 330 | 30 | 40 | 2.5 | 90.0 | 7.5 | 1.179 | <0.05 | 0.067 | 0.693 | 0.107 |
| 1d | 320 | 30 | 40 | 2.5 | 90.0 | 7.5 | 1.180 | <0.05 | 0.104 | 0.643 | 0.093 |
| 1e | 310 | 30 | 40 | 2.5 | 90.0 | 7.5 | 1.159 | <0.05 | 0.155 | 0.589 | 0.082 |
| 1f | 300 | 30 | 40 | 2.5 | 90.0 | 7.5 | 1.183 | <0.05 | 0.213 | 0.552 | 0.072 |
| 1g | 310 | 30 | 40 | 2.5 | 90.0 | 7.5 | 1.218 | <0.05 | 0.147 | 0.664 | 0.083 |
| 1h | 320 | 30 | 40 | 2.5 | 90.0 | 7.5 | 1.230 | <0.05 | 0.096 | 0.804 | 0.106 |
| 1i | 330 | 30 | 40 | 2.5 | 90.0 | 7.5 | 1.236 | <0.05 | 0.062 | 0.912 | 0.123 |
| 1j | 340 | 30 | 40 | 2.5 | 90.0 | 7.5 | 1.194 | <0.05 | 0.039 | 1.050 | 0.140 |
| 1k | 350 | 30 | 40 | 2.5 | 90.0 | 7.5 | 1.216 | <0.05 | 0.038 | 1.225 | 0.156 |

The sulfur feed and exit gas concentration were measured on Agilent 7890A GC system equipped with an OI 5380 pulsed flame photometric detector (PFPD). The detection limit for SO₂ was approximately 0.05 ppm. 40 Nl/h corresponds to 40 liters/hour gas, evaluated at 0° C. and 1 atmosphere pressure.

The results of the tests show that SO₂ to a high degree has been converted to H₂S but with some formation of di-methyl sulfide (DMS) and carbonyl sulfide (COS) as well. For all tests the concentration of SO₂ in the exit gas was below the detection limit of 0.05 ppm showing the desired result of the NiMo based hydrogenation catalyst.

### Example 2

Experiments have been carried out in a laboratory fixed bed reactor at isothermal condition to test the conversion of di-methyl-sulfide (DMS) to H₂S. The fixed bed reactor is placed in an electrically-heated oven and heated to the desired operating temperature. Two internal thermocouples measure the inlet and exit temperatures in the catalytic bed. The oven is equipped with external thermocouples controlling the temperature zones in the oven. These zones are controlled by internal thermocouple readings to obtain isothermal reaction condition in the fixed bed.

The first hydrogenation catalyst material is placed in a SilcoNert 2000™ coated stainless steel reactor on a grid and the reactor aligned to be in the center of the electrical oven. Feed gases are mixed from gas cylinders using mass flow controllers controlling feed of the individual gases. These include nitrogen (N₂), hydrogen (H₂), carbon dioxide (CO₂), and di-methyl-sulfide (DMS).

The first hydrogenation catalyst material used is NiMo-impregnated alumina 1/10″ trilobe extrudates. In the individual test, the catalyst was mixed before loading with catalytically inert carborundum 46, mesh 60 to give a loading height of 110 mm. The catalyst dilution was carried out to limit the chemical conversion such that catalytic activity data could be acquired.

DMS was added to the feed gas to obtain 30 ppm in the feed gas. The sulfur feed and exit gas concentration were measured on an Agilent 8355 S with a S chemiluminescence detector. The peaks to determine H₂S and COS were within the same retention time, making it impossible to distinguish between the two; these are given as one accumulated concentration. Table 2 lists the different test conditions after the NiMo-catalyst has been pre-sulfided for 400 hours.

TABLE 2
Load of 0.5 g NiMo/alumina extrudates and sulfided
for 400 hours with 30 ppm CH₃SCH₃ (DMS) in the gas

| | Conditions | | | Feed gas composition | | | | Exit gas measured | | | |
|---|---|---|---|---|---|---|---|---|---|---|---|
| | T, | P, | Flow, | N₂, | CO₂, | H₂, | DMS, | H₂S | COS | CH₃SH | DMS |
| Test# | ° C. | barg | Nl/h | vol % | vol % | vol % | ppmᵥ | ppm | ppm | ppm | ppm |
| 2a | 350 | 30 | 38 | 2.9 | 91.9 | 5.2 | 29 | 27.5 | 1.4 | 0.15 | 0.03 |
| 2b | 325 | 30 | 37 | 3.0 | 94.3 | 2.7 | 30 | 28.4 | 1.3 | 0.21 | 0.05 |
| 2c | 300 | 30 | 37 | 3.0 | 94.3 | 2.7 | 30 | 27.9 | 1.1 | 0.69 | 0.33 |
| 2d | 350 | 30 | 37 | 3.0 | 94.3 | 2.7 | 30 | 27.5 | 2.3 | 0.09 | 0.03 |
| 2e | 350 | 30 | 38 | 2.9 | 91.9 | 5.2 | 29 | 27.8 | 1.0 | 0.11 | 0.03 |

The results of the tests show that DMS to a high degree was converted to H₂S but with some formation of methylmercaptan (CH₃SH) and carbonyl sulfide (COS) as well. For all tests the DMS concentration in the exit gas correspond to >99.9% conversion except for the lowest tested operating temperature of 300° C. where the DMS conversion was 98.9%. Thus, the desired result of the NiMo based hydrogenation catalyst is shown.

### Example 3

The oxygen hydrogenation activity was investigated for a O2Xtract™ hydrogenation catalyst from Topsoe, comprising Pd and Pt as the active components. The oxygen hydrogenation activity was measured in a CO₂ rich feed gas comprising 2.5 vol % H₂, 2,000 ppm O₂ and either 0 or 10 ppm SO₂. The catalyst space velocity was 180,000 Nm³/h/m³ and the temperature was varied in the range 50-350° C. O₂ concentrations were measured at the inlet and outlet of the catalyst with a dedicated O₂ in CO₂ sensor and O₂ hydrogenation conversion was based on these concentrations. The conversions as a function of catalyst temperature are seen in FIG. 4. It is evident that the catalyst is very active in O₂ hydrogenation, but also that the catalyst activity is significantly hampered by the presence of SO₂. To be able to operate the oxygen hydrogenation catalyst without risking sulfur poisoning, the temperature should be higher than 200° C. and preferably closer to 250-350° C. or above.

### Example 4

In FIG. 3, a layout with the O₂ hydrogenation catalyst, in direct fluid communication with the (first) sulfur hydrogenation catalyst is shown. With 0.5 vol % O₂ in the CO₂ and 3 vol % H₂ added to the CO₂-rich feed gas, the adiabatic temperature increase due to the exothermic O₂ hydrogenation reaction is ~55° C. The (first) sulfur hydrogenation catalyst is preferably operated close to 350° C. and thus the CO₂-rich feed at the inlet to the (second) oxygen hydrogenation catalyst must be 295° C., which represent a good fit with the temperature range for the oxygen hydrogenation catalyst, where the catalyst is not sensitive for SO₂ poisoning.

With 1 vol % O₂ in the CO₂-rich feed and 4 vol % H₂ added to the CO₂-rich feed gas, the adiabatic temperature increase due to the O₂ hydrogenation is 110° C. and thus the oxygen hydrogenation catalyst would operate with an inlet temperature of 240° C. to provide the desired 350° C. inlet temperature to the downstream sulfur hydrogenation catalyst. The 240° C. temperature for the oxygen hydrogenation catalyst is in the low end in which case, it may be better to operate with a higher inlet temperature to the oxygen hydrogenation catalyst and cool the process gas between the outlet of the oxygen hydrogenation catalyst and the inlet of the sulfur hydrogenation catalyst. Another alternative is to increase the amount of H₂ to the CO₂-rich gas to decrease the adiabatic temperature increase, but that comes with an increased risk of formation of undesired side reactions in the sulfur hydrogenation catalyst.

The sulfur guard material can be positioned directly downstream the sulfur hydrogenation catalyst (20) or somewhere downstream heat exchanger X1 as seen in FIG. 3. Heat exchanger X1 can preferably be a feed/effluent heat exchanger, where the cold side is positioned upstream the oxygen hydrogenation catalyst and the hot side is positioned as shown in FIG. 3, i.e. at a position downstream the sulfur hydrogenation catalyst.

The present invention has been described with reference to a number of aspects and figures. However, the skilled person is able to select and combine various aspects within the scope of the invention, which is defined by the appended claims. All documents referenced herein are incorporated by reference.

1. A process for cleaning a CO₂-rich gas feed, said CO₂-rich gas feed comprising at least 80 wt % CO₂ and one or more sulfur-containing impurities; wherein said process comprises the steps of:
passing the CO₂-rich gas feed together with a hydrogen-rich feed over a first catalyst active in hydrogenation, whereby at least a portion of the sulfur-containing impurities in the CO₂-rich gas feed is converted to H₂S, to thereby provide a H₂S-containing CO₂ gas stream,
passing the H₂S-containing CO₂ gas stream over one or more guard material, and adsorbing H₂S and optionally one or more sulfur-containing compounds on a guard material, to provide a cleaned CO₂-rich gas stream.

2. The process according to claim 1, wherein the CO₂-rich gas feed comprises at least 90 wt % CO₂.

3. The process according to claim 1, wherein the one or more sulfur-containing impurities within said CO₂-rich gas feed are selected from organosulfur compounds.

4. The process according to claim 1, wherein the one or more sulfur-containing compounds adsorbed onto the guard material are selected from thiols, sulfides, disulfides, sulfones, sulfoxides and thioketones and COS.

5. The process according to claim 1, wherein the guard material is active in adsorption of H₂S.

6. The process according to claim 1, comprising the steps of
optionally adjusting the temperature and/or water content of the H₂S-containing CO₂ gas stream
passing the H₂S-containing CO₂ gas stream over a first guard material, followed by
optionally adjusting the temperature and/or water concentration of the H₂S-containing CO₂ gas stream, followed by
passing the H₂S-containing CO₂ gas stream over a second guard material, and adsorbing one or more sulfur-containing compounds on said first and said second guard materials, to provide a cleaned CO₂-rich gas stream.

7. The process according to claim 6, wherein the first guard material is a ZnO or Cu-promoted ZnO guard material, and wherein the second guard material is a ZnO, Cu-promoted ZnO or Cu—Zn—Al guard material.

8. The process according to claim 1, wherein the first catalyst active in hydrogenation comprises a CoMo/NiMo catalyst.

9. The process according to claim 1, further comprising a step of removing water from the H₂S-containing CO₂ gas stream and/or adjusting the temperature of the H₂S-containing CO₂ gas stream, prior to passing it over said guard material.

10. The process according to claim 9, wherein the step of removing H₂O comprises cooling the H₂S and H₂O containing CO₂-rich gas feed and separating condensed water.

11. The process according to claim 1, wherein the first hydrogenation catalyst material is located within a reactor vessel, said reactor vessel being arranged to receive said CO₂-rich gas feed and said hydrogen-rich feed, optionally in admixture.

12. The process according to claim 1, wherein the sulfur containing impurity is SO₂ and the concentration of SO₂ in the CO₂-rich gas feed at the inlet of said reactor vessel is 0.1-100 ppm SO₂.

13. The process according to claim 1, wherein the CO₂-rich gas feed additionally comprises oxygen, and wherein process further comprises the step of passing the CO₂-rich gas feed together with a hydrogen-rich feed over a second catalyst active in oxygen hydrogenation, and being positioned upstream the first catalyst, thereby converting at least a portion of the oxygen in the CO₂-rich gas feed to H₂O.

14. The process according to claim 1, wherein the total content of O₂ in the CO₂-rich gas feed is 0.1-10,000 ppm O₂.

15. The process according to claim 1, wherein the total content of H₂ in the combined feed of the CO₂-rich gas feed and the hydrogen-rich feed, after mixing of said feeds and carrying out sulfur and/or oxygen hydrogenation, is 0.2-10 vol % H₂.

16. The process according to claim 1, wherein the total content of H₂O in the combined feed of the CO₂-rich gas feed and the hydrogen-rich feed, after mixing of said feeds and passing the gas mixture over the hydrogenation catalyst(s), is no more than 10 vol %.

17. The process according to claim 1, wherein the cleaned CO₂-rich gas stream has a sulfur in all forms content of less than 500 ppb.

18. The process according to claim 13, wherein the cleaned CO₂-rich gas stream has an oxygen content of less than 200 ppm.

19. The process according to claim 1, wherein the operating temperature of the first catalyst active in hydrogenation is in the range 250-450° C., the operating temperature of the optional oxygen hydrogenation catalyst is in the 200-400° C. range and the operating temperature of the guard material is in the range 80-450° C.

20. The process according to claim 1, wherein the CO₂ feed is derived from a renewable source.

21. A process for production of a syngas stream, said process comprising the process according to claim 1, and further comprising:
providing at least a portion of said cleaned CO₂-rich gas stream;
providing a second hydrogen-rich feed, optionally obtained from the process of electrolysis of water in one or more electrolysis unit(s);
mixing at least a portion of said portion of the cleaned CO₂-rich stream feed with the second hydrogen-rich feed, to provide at least one syngas feed stream;
passing the syngas feed stream over one or more catalyst(s) active in converting CO₂ and H₂ into a mixture of CO, H₂O, H₂ and CO₂.

22. A process for production of a synthetic fuel stream, said process comprising the process according to claim 21, and further comprising the step of converting said at least one syngas stream to at least one synthetic fuel stream, a DME stream, or a synthetic fuel stream.
